(19) United States
(12) Patent Application Publication
(10) Pub. No.: US 2026/0165931 A1
(43) Pub. Date: Jun. 18, 2026

(54) Aqueous Make-up-Removing Composition Having a Mild Peeling Effect

(71) Applicant: L'OREAL, PARIS (FR)

(72) Inventors: ESTELLE MATHONNEAU, SAINT-OUEN (FR); MAUD RIBADEAU DUMAS, CHEVILLY-LARUE (FR); SIRINE HNANA, CHEVILLY-LARUE (FR); BÉATRICE BINUTTI, CHEVILLY-LARUE (FR)

(73) Assignee: L'OREAL, Paris (FR)

(21) Appl. No.: 19/123,473

(22) PCT Filed: Oct. 24, 2023
(86) PCT No.: PCT/EP2023/079547

§ 371 Date: Apr. 23, 2025

(30) Foreign Application Priority Data
Oct. 24, 2022 (FR) 2211013

(51) Int. Cl. A61K 8/37; A61K 8/03; A61K 8/34; A61K 8/44; A61Q 1/14

(52) U.S. Cl. CPC A61K 8/375; A61K 8/03; A61K 8/345; A61K 8/44; A61Q 1/14; A61K 2800/26; A61K 2800/28; A61K 2800/805

(57) ABSTRACT

The present invention relates to a make-up-removing aqueous cosmetic composition, in particular a micellar water, having a mild peeling effect, comprising an aqueous phase comprising: at least one polyhydroxy acid, at least one hydroxy acid chosen from a-hydroxy acids and/or [3-hydroxy acids, at least one amphoteric surfactant, preferably of betaine type, and at least one non-ionic surfactant, such as a poloxamer. It also relates to a method for removing make-up and/or for cleansing keratin materials using such a composition.

The present invention relates to a make-up-removing aqueous cosmetic composition, in particular a micellar water, having a mild peeling effect, containing a combination of polyhydroxy acid(s) (PHA(s)) and of hydroxy acid(s) (AHA(s)/BHA(s)), at least one amphoteric surfactant, preferably of betaine type, and at least one non-ionic surfactant.

Removing make-up from the skin is very important for caring for keratin materials, in particular the skin, the face, the eyelids or the lips, but also keratin fibres, such as the eyelashes. It must be as efficient as possible because fatty residues, such as excess sebum, residues of cosmetics used daily and make-up products, especially water-resistant ones, accumulate in particular in skin folds and at the surface of the skin, and can clog the pores of the skin and thus cause the appearance of pimples.

More particularly, the sensitivity of the eye and eyelids and the nature of certain eye shadows (waterproof eye shadows) require specially adapted make-up-removing formulations to guarantee both good ocular tolerance and the maintenance of the skin barrier, at the same time as easy and effective make-up removal.

The majority of the make-up-removing compositions available are compositions comprising oils, the latter making it possible to dissolve the fatty residues and to disperse the residual pigments. Mention may in particular be made of two-phase compositions, i.e. consisting of two distinct phases, in particular of an aqueous phase and of an oily phase, which require prior agitation before application. Such formulations make possible good removal of make-up and are well tolerated but leave an often greasy finish on the skin.

There also exist micellar waters, which are fluid single-phase compositions which confer freshness on application. Such compositions are not always effective with regard to removing make-up from and cleansing the skin, and generally do not exhibit any additional benefit, in particular with regard to cell renewal.

There thus remains a need for effective and well-tolerated make-up-removing compositions, which exhibit a liquid and fresh texture, and which provide a benefit on application, and in particular with regard to cell renewal, while respecting sensitive skin.

Peels are a well-known means for accelerating cell renewal, improving the surface appearance of the skin, in particular for treating or smoothing visible and/or tactile irregularities of human skin. These peels have the effect of removing a part of the skin to be treated (epidermis and possibly superficial layer of the dermis), by the application of compositions containing high concentrations of agents stimulating the desquamation of the skin, such as hydroxy acids, for example glycolic acid or salicylic acid. Although giving satisfactory results in terms of peeling effect, the products used for peels generally contain acids at a high concentration giving compositions of pH<2, and for this reason result in significant disadvantages on application and after application: redness, tingling, burning sensation.

Some foaming cleansing products claim a neutral pH (7) and a peeling effect, but the peeling effect proves to be insufficient because a neutral pH (7) is generally not compatible with the effectiveness of the hydroxy acids used as peeling active principles. Moreover, these foaming products contain surfactants, and require rinsing, which can result in skin dryness, or even aggravate a possible skin intolerance. There thus remains a need for novel types of compositions which are effective both in terms of removal of make-up and of cell renewal, and exhibit good skin and ocular tolerance compatible with the removal of make-up from the eyes.

The Applicant Company has discovered, surprisingly, that an aqueous composition comprising the combination of at least one polyhydroxy acid (PHA), of at least one hydroxy acid (AHA/BHA) and of at least one amphoteric surfactant, preferably of betaine type, combined with the presence of at least one non-ionic surfactant, such as a poloxamer, makes possible both effective removal of make-up and effective cell renewal, with very good ocular tolerance and good maintenance of the skin barrier.

A subject-matter of the present invention is thus a liquid aqueous composition, preferably a micellar water, which removes make-up from and/or cleanses keratin materials, comprising: at least one polyhydroxy acid (PHA), at least one hydroxy acid chosen from α-hydroxy acids (AHAs) and/or β-hydroxy acids (BHAs), at least one amphoteric surfactant, preferably of betaine type, at least one non-ionic surfactant, and water.

Advantageously, the composition according to the invention is an aqueous single-phase one, and exhibits a fluid and transparent appearance similar to water under ambient conditions of temperature (25° C.) and pressure (100 kPa).

Another subject-matter of the present invention is a method for removing make-up from and/or for cleansing keratin materials, preferably skin and/or keratin fibres, such as the eyelashes, comprising the application, to the keratin materials, of a composition according to the invention.

The term “keratin materials” is understood to mean the skin, mucous membranes and/or superficial body growths. Preferably, the keratin materials are the skin, in particular the skin of the face, the mucous membranes, such as the lips, the eyelids or the outline of the eye, and/or the superficial body growths, such as the eyelashes.

The components of the composition according to the invention are now described in more detail.

### Aqueous Phase

The composition according to the invention comprises a physiologically acceptable aqueous phase. The term “physiologically acceptable” is understood to mean a medium compatible with keratin materials.

The composition according to the invention preferably comprises an aqueous phase comprising water, in particular at least 50% of water, and optionally one or more organic solvent(s) soluble in water, at 25° C. This (These) solvent(s) can advantageously be chosen, for example, from polyols having in particular from 2 to 20 carbon atoms, preferably from 2 to 6 carbon atoms, such as glycerol, diglycerol, propylene glycol, isoprene glycol, dipropylene glycol, butylene glycol, hexylene glycol, 1,3-propanediol, pentylene glycol, polyethylene glycols having from 2 to 200 ethylene oxide units and their mixtures.

The composition generally comprises from 50% to 99% by weight of water, with respect to the total weight of the composition, preferably from 70% to 98% by weight, preferably from 80% to 98% by weight, preferably from 85% to 97% by weight, of water, with respect to the total weight of the composition.

The amount of organic solvent(s) can range, for example, from 0.01% to 15% by weight, preferably from 0.5% to 13% by weight, better still from 1% to 10% by weight, with respect to the total weight of the composition.

The aqueous phase of the composition according to the invention also comprises: at least one polyhydroxy acid (PHA), at least one hydroxy acid chosen from α-hydroxy acids (AHAs) and/or β-hydroxy acids (BHAs), at least one amphoteric surfactant, preferably of betaine type, at least one non-ionic surfactant.

### Polyhydroxylated Acids or Polyhydroxy Acids or PHAs

The composition according to the present invention comprises at least one first compound chosen from polyhydroxylated acids, and their derivatives in the form of salts or their lactone forms.

The term “polyhydroxylated acid” designates an organic compound which possesses at least one carboxyl group and a plurality of hydroxyl groups. The number of carboxyl groups in the polyhydroxylated acid is not limited but one or two carboxyl groups are preferred, one carboxyl group being more preferable. The number of hydroxyl groups in the polyhydroxy acid is not limited either but 2 to 10 are preferred, and 2 to 6 are even more preferred. The number of carbon atoms in the polyhydroxy acid is not limited but preferably the PHA contains from 3 to 11 carbon atoms, preferably from 3 to 8.

The PHA can be aliphatic or aromatic. In other words, the polyhydroxylated acid may be chosen from aliphatic polyhydroxylated acids or aromatic polyhydroxylated acids. Preferably, the aliphatic polyhydroxylated acid is chosen from sugar acids.

The polyhydroxylated acid is advantageously chosen from: dihydroxypropanoic acid, such as glyceric acid; trihydroxybutanoic acid, such as erythronic acid and threonic acid; tetrahydroxypentanoic acid, such as ribonic acid, arabinonic acid, xylonic acid and lyxonic acid; pentahydroxyhexanoic acid, such as allonic acid, altronic acid, gluconic acid, mannonic acid, gulonic acid, idonic acid, galactose, galactonic acid and talonic acid; hexhydroxyheptanoic acid, such as glucoheptanoic acid or galactoheptonic acid; lactobionic acid, maltobionic acid, their derivatives in the form of salts or their lactone forms, such as gluconolactone or ribonolactone, and their mixtures.

The type of salt is not limited. Examples of salts comprise alkali metal salts, such as the sodium salt and the potassium salt; alkaline earth metal salts, such as the calcium salt and the magnesium salt; zinc salts; iron salts; ammonium salts; amine salts, such as the monoethanolamine salt, the diethanolamine salt and the triethanolamine salt; and their mixtures. The sodium salt is preferable. If two or more acid salts are used, they can be identical or different.

Advantageously, the polyhydroxy acid is provided in the form of a lactone. The lactone ring is preferably saturated and preferably, in this case, the polyhydroxy acid is chosen from gluconolactone, ribonolactone and their mixtures.

The list of PHAs which can be used according to the invention also incorporates galactose; although it is a hexose of the family of the aldoses, it is pentahydroxylated and can be used as a mild exfoliant.

Preferably, the polyhydroxy acid according to the invention is thus chosen from: lactobionic acid, galactose, gluconic acid, gluconolactone and their mixtures. Advantageously, the aqueous composition according to the invention uses just one or else several PHAs, so as to select or combine their effects. For example, lactobionic acid, derived from lactose, is moisturizing, anti-oxidizing and soothing. Galactose, which is naturally found in the deep layers of the skin, has an action on the production of collagen. Gluconic acid, gluconolactone, like galactose, are naturally present in skin cells. Their role is as antioxidant and anti-inflammatory.

Preferably, said polyhydroxy acid used in the composition according to the invention is gluconolactone.

The amount of PHA in the composition according to the present invention is within the range from 0.1% to 5%, preferably from 0.2% to 4%, preferably from 0.5% to 3%, preferably from 1% to 2%, by weight (of active material), with respect to the total weight of the composition.

### Hydroxy Acids

In addition, the composition according to the invention comprises at least one hydroxy acid different from the PHA(s), chosen from α-hydroxylated acids, β-hydroxylated acids and their mixtures.

### α-Hydroxy Acids (Also Called AHAs or α-Hydroxylated Acids)

The term “α-hydroxylated acid” is understood to mean a carboxylic acid having at least one hydroxyl functional group occupying an a position on said acid (carbon adjacent to a carboxylic acid functional group).

The α-hydroxy acids are chosen in particular from glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid; mandelic acid; phenyllactic acid; gluconic acid; galacturonic acid; aleuritic acid; tartronic acid; tartaric acid; malic acid; fumaric acid; their esters, their salts and their mixtures. Mixtures of these different acids can also be used.

Advantageously, the α-hydroxy acid(s) is (are) chosen from glycolic acid, lactic acid, citric acid, mandelic acid, malic acid, tartaric acid, their esters, their salts and their mixtures. Thus, glycolic acid (which results from sugar cane) is one of the most effective AHAs because its smaller molecule makes it possible for it to exfoliate more deeply into the skin. It refines the skin texture and gives radiance to the complexion. Glycolic acid would also have a slight decreasing effect on the production of sebum. Lactic acid originates from the fermentation of milk but it is also found in several fruits. It is one of the mildest acids; thus it is ideal for dry and sensitive skin. It is an excellent natural humectant. It attracts water and retains it in the skin. As regards malic acid, it is found in particular in apples, pears and grapes. It is often used in cosmetic preparations to rebalance their pH but on the skin, moisturizing properties are also attributed to it and it also helps to stimulate the production of collagen. It can penetrate very deeply into the skin by virtue of its small molecular size. Mandelic acid, originating from almonds, is one of the mildest AHAs because its molecular size is greater than that of the other acids and thus limits its ability for absorption deeply into the skin. It acts instead at the surface's and is thus less irritating than other acids acting more intensely. It also helps to reduce hyperpigmentation and to homogenize the complexion. Tartaric acid originates in particular from grapes. This acid has antioxidant and anti-inflammatory properties. Citric acid originates from citrus fruits. It is mainly used for its lightening properties with regard to brown spots and hyperpigmentation. It is also astringent and can thus also reduce the size of the pores. Advantageously, the aqueous composition according to the invention uses several AHAs so as to combine their effects.

More particularly, the α-hydroxy acid(s) is (are) chosen from glycolic acid, citric acid, lactic acid, their salts, their esters and their mixtures.

The ester derivatives comprise in particular the ester compounds of glycolic acid, of lactic acid, such as methyl lactate, ethyl lactate or butyl lactate, and likewise the ester compounds of glycolic acid, of tartaric acid, of mandelic acid, of citric acid.

Particularly suitable α-hydroxylated acids in the composition of the present invention are glycolic acid, lactic acid and their mixtures.

Preferably, the α-hydroxylated acid in the aqueous composition of the present invention is glycolic acid.

### β-Hydroxy Acids (Also Known as BHAs or β-Hydroxylated Acids)

The term “β-hydroxy acid” is understood to mean, within the meaning of the present invention, a carboxylic acid having a hydroxyl functional group and a carboxyl functional group separated by two carbon atoms.

The β-hydroxy acid(s) is (are) chosen in particular from: salicylic acid, β-hydroxypropionic acid, β-hydroxybutyric acid, β-hydroxymethylbutyric acid, carnitine, their derivatives and their combinations. Preferably, the β-hydroxy acid is chosen from salicylic acid and its derivatives.

Advantageously, the composition according to the invention comprises at least one compound chosen from salicylic acid and its derivatives.

Salicylic acid, or 2-hydroxybenzoic acid, is an active agent, in particular in combating imperfections.

Alternatively, a salicylic acid derivative is present. The salicylic acid derivatives are preferably those of formula (I):

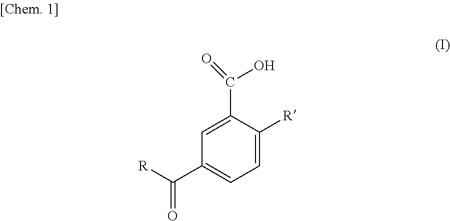

in which:
   the radical R denotes a saturated, linear, branched or cyclic, aliphatic chain having from 2 to 22 carbon atoms; an unsaturated chain having from 2 to 22 carbon atoms containing one or more double bonds which can be conjugated; an aromatic nucleus bonded to the carbonyl radical directly or via saturated or unsaturated aliphatic chains having from 2 to 7 carbon atoms; it being possible for said nuclei and chains to be substituted by one or more substituents, identical or different, chosen from (a) halogen atoms, (b) the trifluoromethyl group, (c) hydroxyl groups in free form or esterified by an acid having from 1 to 6 carbon atoms or (d) a carboxyl group in free form or esterified by a lower alcohol having from 1 to 6 carbon atoms;
   R′ is a hydroxyl group or ester group of formula —O—C(O)—R1 in which R1 denotes a saturated or unsaturated, linear or branched, aliphatic chain containing from 1 to 18 carbon atoms;
   and also their salts resulting from an inorganic or organic base.

Preferentially:
the radical R denotes a saturated, linear, branched or cyclic, aliphatic chain containing from 3 to 11 carbon atoms; an unsaturated chain containing from 3 to 17 carbon atoms and comprising one or more conjugated or non-conjugated double bonds; it being possible for said hydrocarbon chains to be substituted by one or more substituents, identical or different, chosen from (a) halogen atoms, (b) the trifluoromethyl group, (c) hydroxyl groups in free form or esterified by an acid having from 1 to 6 carbon atoms or (d) a carboxyl function in free form or esterified by a lower alcohol having from 1 to 6 carbon atoms;
R′ is a hydroxyl group or ester group of formula —O—C(O)—R1 in which R1 denotes a —(CH₂)ₙ—CH₃ radical where n is a number ranging from 0 to 14;
and also their salts obtained by salification by an inorganic or organic base.

The more particularly preferred compounds are those in which the radical R is a C₃-C₁₁ alkyl group and R′ denotes hydroxyl. Other particularly advantageous compounds are those in which R represents a chain derived from linoleic, linolenic or oleic acid. Another group of particularly preferred compounds consists of compounds in which the radical R denotes a C₃-C₁₁ alkyl group carrying a carboxyl function in free form or esterified by a lower alcohol having from 1 to 6 carbon atoms and R′ denotes hydroxyl.

Mention may thus be made, as preferred salicylic acid derivatives, of 5-(n-octanoyl) salicylic acid (or capryloylsalicylic acid), 5-(n-decanoyl) salicylic acid, 5-(n-dodecanoyl) salicylic acid or 5-(n-heptyloxy) salicylic acid, and their corresponding salts.

Preferably, the β-hydroxy acid is salicylic acid.

Advantageously, salicylic acid, or 2-hydroxybenzoic acid, is used as sole hydroxy acid or else in combination with an AHA in order to ensure increased penetration of the composition into the skin.

The salts of these hydroxy acids are also considered within the meaning of the invention. The salts can be obtained by salification of the acid under consideration with an organic or inorganic base. Mention may in particular be made, as inorganic bases, of alkali metal or alkaline earth metal hydroxides, such as, for example, sodium hydroxide or potassium hydroxide, and aqueous ammonia. As regards the organic bases, they can in particular be bases of amine or alkanolamine type.

The hydroxy acid(s) is (are) present in the composition of the present invention according to a content ranging from 0.001% to 10% by weight, preferably from 0.01% to 5% by weight, preferably from 0.1% to 3% and better still from 0.1% to 2%, preferably from 0.1% to 1.5%, preferably from 0.1% to 1%, preferably from 0.2% to 0.8%, by weight (of active material), with respect to the total weight of the composition.

### Amphoteric Surfactants

The composition according to the invention also comprises one or more amphoteric surfactants. The amphoteric surfactants capable of being used in the invention can be optionally quaternized secondary or tertiary aliphatic amine derivatives, in which the aliphatic group is a linear or branched chain comprising from 8 to 22 carbon atoms, said amine derivatives containing at least one anionic group, such as, for example, a carboxylate, sulfonate, sulfate, phosphate or phosphonate group.

Mention may in particular be made of betaines and sulfobetaines (or sultaines), and also their mixtures, and in particular, alone or as a mixture, of: betaine, (C₈-C₂₀) alkyl betaines, and in particular coco betaine; (C₈-C₂₀) alkylamido (C₁-C₆)alkyl betaines, and in particular (C₈-C₂₀) alkylamidopropyl betaines, such as cocoamidopropyl betaine, (C₈-C₂₀)alkyl sulfobetaines.

Mention may also be made, among the optionally quaternized secondary or tertiary aliphatic amine derivatives capable of being employed, of the products with the following respective structures (A1) and (A2):

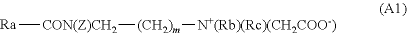

in which: Ra represents a C₁₀-C₃₀ alkyl or alkenyl group derived from an acid Ra—COOH preferably present in hydrolysed coconut oil, a heptyl, nonyl or undecyl group; Rb represents a β-hydroxyethyl group; Rc represents a carboxymethyl group; m is equal to 0, 1 or 2; Z represents a hydrogen atom or a hydroxyethyl or carboxymethyl group;

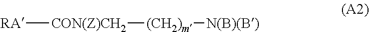

in which: B represents —CH₂CH₂OX′, with X′ representing —CH₂—COOH), —CH₂—COOZ′, —CH₂CH₂—COOH), —CH₂CH₂—COOZ′ or a hydrogen atom; B′ represents —(CH₂)_{z}—Y′, with z=1 or 2, and Y′ representing —COOH, —COOZ′, —CH₂—CHOH—SO₃H or —CH₂—CHOH—SO₃Z′; m′ is equal to 0, 1 or 2; Z represents a hydrogen atom or a hydroxyethyl or carboxymethyl group; Z′ represents an ion resulting from an alkali metal or alkaline earth metal, such as sodium, potassium or magnesium; an ammonium ion; or an ion resulting from an organic amine and in particular from an aminoalcohol, such as mono-, di- or triethanolamine, mono-, di- or triisopropanolamine, 2-amino-2-methyl-1-propanol, 2-amino-2-methyl-1,3-propanediol and tris(hydroxymethyl)aminomethane; Ra′ represents a C₁₀-C₃₀ alkyl or alkenyl group of an acid Ra′COOH preferably present in hydrolysed linseed oil or coconut oil, an alkyl group, in particular a C₁₇ alkyl group, and its iso form, an unsaturated C₁₇ group.

The compounds corresponding to the formula (A2) are particularly preferred.

Mention may be made, among the compounds of formula (A2) for which X′ represents a hydrogen atom, of the compounds known under the (CTFA) names sodium cocoamphoacetate, sodium lauroamphoacetate, sodium caproamphoacetate and sodium capryloamphoacetate. Other compounds of formula (A2) are known under the (CTFA) names disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroampho-dipropionic acid and cocoamphodipropionic acid.

Use may also be made of the compounds of formula (A3):

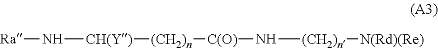

in which: Ra″ represents a C₁₀-C₃₀ alkyl or alkenyl group of an acid Ra″—C(O)OH, preferably present in hydrolysed linseed oil or coconut oil; Y″ represents the —C(O)OH, —C(O)OZ″, —CH₂—CH(OH)—SO₃H or —CH₂—CH(OH)—SO₃—Z″ group with Z″ representing a cation resulting from an alkali metal or alkaline earth metal, such as sodium, an ammonium ion or an ion resulting from an organic amine; Rd and Re, independently of each other, represent a C₁-C₄ alkyl or hydroxyalkyl radical; and n and n′, independently of each other, denote an integer ranging from 1 to 3.

Mention may in particular be made, among the compounds of formula (A3), of the compound classified in the CTFA dictionary under the name sodium diethylaminopropyl cocoaspartamide.

### Amphoteric Surfactants of Betaine Type

The composition according to the invention preferably comprises at least one amphoteric surfactant chosen from betaines. The amphoteric or zwitterionic surfactants of betaine type of the invention are preferably chosen from:
alkyl betaines, in particular lauryl betaine such as, for example, the product Genagen KB® from Clariant, or coco betaine, such as, for example, the product Dehyton AB 30® from BASF or TEGO Betain AB 1214 from Evonik Goldschmidt GmbH;
N-alkylamido betaines and their derivatives, in particular cocamidopropyl betaine (for example Lebon 2000 HG® from Sanyo or Empigen BB® from Albright and Wilson or Dehyton PK 45), lauramidopropyl betaine (for example Rewoteric AMB12P® from Witco);
sultaines, such as cocoylamidopropyl hydroxysulfobetaine (for example, Crosultaine C-500 from Croda);
and their mixtures.

Preferably, the amphoteric surfactant of betaine type is chosen from alkyl betaines, such as coco betaine and/or lauryl betaine, and N-alkylamido betaines, such as cocamidopropyl betaine; sultaines, such as cocoylamidopropyl hydroxysulfobetaine; and their mixtures.

Preferably, the amphoteric surfactant of betaine type is chosen from alkyl betaines and N-alkylamido betaines, preferably from N-alkylamido betaines, more preferentially from cocamidopropyl betaine, coco betaine and their mixtures.

These preferred amphoteric surfactants of betaine type, preferably chosen from alkyl betaines, have the advantage of being able to resist low pH (even below pH 6, such as pH 4.8) without any turbidity, even at low temperatures, such as below 15° C., in particular at 12° C. or even at 4° C., thus guaranteeing the transparency of the present formula (<20 NTU) whatever the storage conditions.

The content as active material of amphoteric surfactant(s) chosen from betaine type, in particular chosen from alkyl betaines, is preferably within the range from 0.1% to 10% by weight, preferably from 0.1% to 5% by weight, preferably from 0.1% to 3% by weight, preferably from 0.1% to 2% by weight, preferably from 0.1% to 1% by weight, preferably from 0.1% to 0.5% by weight of active material, with respect to the total weight of the composition.

### Non-Ionic Surfactants

The composition comprises one or more non-ionic surfactants.

They can be chosen from alcohols, α-diols and (C₂₀)alkylphenols, these compounds being polyethoxylated and/or polypropoxylated and/or polyglycerolated, it being possible for the number of ethylene oxide and/or propylene oxide groups to range from 1 to 100 and it being possible for the number of glycerol groups to range from 2 to 30, or else these compounds comprising at least one fatty chain comprising from 8 to 30 carbon atoms, in particular from 16 to 30 carbon atoms.

Mention may also be made of non-ionic surfactants of alkyl (poly)glycoside type, in particular represented by the following general formula: R1O—(R2O)ₜ-(G)ᵥ, in which: R1 represents a linear or branched alkyl or alkenyl radical comprising from 6 to 24 carbon atoms, in particular from 8 to 18 carbon atoms, or an alkylphenyl radical, the linear or branched alkyl radical of which comprises from 6 to 24 carbon atoms, in particular from 8 to 18 carbon atoms; R2 represents an alkylene radical comprising from 2 to 4 carbon atoms; G represents a sugar unit comprising from 5 to 6 carbon atoms; t denotes a value ranging from 0 to 10, preferably from 0 to 4; and v denotes a value ranging from 1 to 15, preferably from 1 to 4.

Preferably, the alkyl(poly)glycoside surfactants are compounds of the formula described above, in which: R1 denotes a saturated or unsaturated and linear or branched alkyl radical comprising from 8 to 18 carbon atoms; R2 represents an alkylene radical comprising from 2 to 4 carbon atoms; t denotes a value ranging from 0 to 3, preferably equal to 0; G denotes glucose, fructose or galactose, preferably glucose; it being possible for the mean degree of polymerization, that is to say the value of v, to range from 1 to 15, preferably from 1 to 4; the mean degree of polymerization being more particularly of between 1 and 2.

The glycoside bonds between the sugar units are generally of 1-6 or 1-4 type, preferably of 1-4 type. Preferably, the alkyl (poly)glycoside surfactant is an alkyl (poly)glucoside surfactant. Preference is very particularly given to C₈/C₁₆ alkyl (poly)glucosides of 1-4 type and in particular decyl glucosides and caprylyl/capryl glucosides, lauryl glucoside and coco glucoside.

Mention may be made, among the commercial products, of the products sold by BASF under the names Plantaren® (600 CS/U, 1200 and 2000) or Plantacare® (818, 1200 and 2000); the products sold by SEPPIC under the names Oramix CG 110 and Oramix® NS 10; the products sold by BASF under the name Lutensol GD 70 or also the products sold by Chem Y under the name AG10 LK.

Preferably, use is made of C₈/C₁₆ alkyl (poly)glycosides of 1-4 type, in particular as a 53% aqueous solution, such as those sold by Cognis under the reference Plantacare® 818 UP.

Preferably, the composition according to the invention uses one or more surfactants exhibiting, at 25° C., an HLB (hydrophilic-lipophilic balance), in the Griffin sense, of greater than or equal to 8, more particularly an HLB of greater than or equal to 10, preferably of greater than or equal to 12, preferably of greater than or equal to 13, used alone or as a mixture. The HLB value according to Griffin is defined in J. Soc. Cosm. Chem., 1954 (Volume 5), pages 249-256.

Mention may in particular be made of: monosaccharide esters and ethers, such as the mixture of cetylstearyl glucoside and of cetyl and stearyl alcohols, for example Montanov 68 from SEPPIC; oxyethylenated and/or oxypropylenated ethers (which can comprise from 1 to 150 oxyethylene and/or oxypropylene groups) of glycerol; oxyethylenated and/or oxypropylenated ethers (which can comprise from 1 to 150 oxyethylene and/or oxypropylene groups) of fatty alcohols (in particular of C₈-C₂₄ and preferably C₁₂-C₁₈ alcohols), such as the oxyethylenated ether of cetearyl alcohol comprising 30 oxyethylene groups (CTFA name “Ceteareth-30”), the oxyethylenated ether of stearyl alcohol comprising 20 oxyethylene groups (CTFA name “Steareth-20”) and the oxyethylenated ether of the mixture of C₁₂-C₁₅ fatty alcohols comprising 7 oxyethylene groups (CTFA name “C12-15 Pareth-7”), sold under the name Neodol 25-7® by Shell Chemicals; the esters of fatty acid (in particular of C₈-C₂₄ and preferably C₁₆-C₂₂ acid) and of polyethylene glycol (which can comprise from 1 to 150 ethylene glycol units), such as PEG-50 stearate and PEG-40 monostearate, sold under the name Myrj 52P® by ICI Uniqema; the esters of fatty acid (in particular of C₈-C₂₄ and preferably C₁₆-C₂₂ acid) and of oxyethylenated and/or oxypropylenated glycerol ethers (which can comprise from 1 to 150 oxyethylene and/or oxypropylene groups), such as PEG-200 glyceryl monostearate, sold under the name Simulsol 220® by SEPPIC; polyethoxylated glyceryl stearate comprising 30 ethylene oxide groups, such as the product Tagat S® sold by Goldschmidt, polyethoxylated glyceryl oleate comprising 30 ethylene oxide groups, such as the product Tagat O® sold by Goldschmidt, polyethoxylated glyceryl cocoate comprising 30 ethylene oxide groups, such as the product Varionic LI 13® sold by Sherex, polyethoxylated glyceryl isostearate comprising 30 ethylene oxide groups, such as the product Tagat L® sold by Goldschmidt, and polyethoxylated glyceryl laurate comprising 30 ethylene oxide groups, such as the product Tagat 1® from Goldschmidt; esters of fatty acids (in particular of C₈-C₂₄ and preferably C₁₆-C₂₂ acid) and of oxyethylenated and/or oxypropylenated sorbitol ethers (which can comprise from 1 to 150 oxyethylene and/or oxypropylene groups), commonly known as “carbohydrate-based esters (CBEs)”. Examples, but not limited, of said carbohydrate-based esters are polysorbate 20, sold under the name Tween 20® by Croda, polysorbate 21, sold under the name Tween 21® by Croda, or polysorbate 60, sold under the name Tween 60® by Croda; dimethicone copolyol, such as that sold under the name Q2-5220® by Dow Corning; dimethicone copolyol benzoate (Finsolv SLB 101® and 201® from Fintex). Examples of such surfactants are provided in the document US 2012/042894, which is incorporated here by way of reference; poloxamers, which are copolymers of propylene oxide and of ethylene oxide.

Said copolymers of propylene oxide (PO) and of ethylene oxide (EO), also called EO/PO polycondensates, are copolymers consisting of polyethylene glycol and polypropylene glycol blocks. Preferably, the EO/PO polycondensate is chosen from polyethylene glycol/polypropylene glycol/polyethylene glycol triblock polycondensates, for example those having the following chemical structure:

H—(O—CH₂—CH₂)ₐ—(O—CH(CH₃)—CH₂)_{b}—(O—CH₂—CH₂)ₐ—OH, in which formula a ranges from 2 to 150 and b ranges from 1 to 100; preferably, a ranges from 10 to 130 and b ranges from 20 to 80.

The ethylene oxide and propylene oxide block copolymer (polycondensate) surfactant preferably has a weight-average molecular weight ranging from 1000 to 20 000, better still ranging from 1500 to 19 000, in particular ranging from 2000 to 18 000 and even better still ranging from 4000 to 17 000.

Mention may in particular be made, as polycondensates, of the polyethylene glycol/polypropylene glycol/polyethylene glycol triblock polycondensates sold under the “Synperonic” names by Unigema, such as the condensates of ethylene oxide, of propylene oxide and of ethylene oxide (13 EO/30 PO/13 EO) (MW: 2900) sold under the name Synperonic PE/L 64 NAA LQ (Poloxamer 184), the condensates of ethylene oxide, of propylene oxide and of ethylene oxide (8 EO/30 PO/8 EO) (MW: 2500) sold under the name Synperonic PE/L 62 (INCI name: Poloxamer 182), the condensates of ethylene oxide, of propylene oxide and of ethylene oxide (6 EO/67 PO/6 EO) (MW: 4400) sold under the name Synperonic PE/L 121 (INCI name: Poloxamer 401), the condensates of ethylene oxide, of propylene oxide and of ethylene oxide (46 EO/16 PO/46 EO) (MW: 5000) sold under the name Synperonic® PE/F38 (INCI name: Poloxamer 108), the condensates of ethylene oxide, of propylene oxide and of ethylene oxide (128 EO/54 PO/128 EO) (MW: 14 000) sold under the name Synperonic® PE/F108 (INCI name: Poloxamer 338), the condensates of ethylene oxide, of propylene oxide and of ethylene oxide (11 EO/21 PO/11 EO) (MW: 2200) sold under the name Synperonic® PE/L44 (INCI name: Poloxamer 124), the condensates of ethylene oxide, of propylene oxide and of ethylene oxide (5 EO/21 PO/5 EO) (MW: 1630) sold under the name Synperonic® PE/L42 (INCI name: Poloxamer 122), the condensates of ethylene oxide, of propylene oxide and of ethylene oxide (98 EO/67 PO/98 EO) (MW: 12 000) sold under the name Synperonic® PE/F127 (INCI name: Poloxamer 407), the condensates of ethylene oxide, of propylene oxide and of ethylene oxide (97 EO/39 PO/97 EO) (MW: 10 800) sold under the name Synperonic® PE/F88 (INCI name: Poloxamer 238) and their mixtures.

Advantageously, the non-ionic surfactant(s) is(are) present in the composition according to an active material content ranging from 0.01% to 10% by weight, preferably from 0.05% to 5% by weight, preferably from 0.05% to 2% by weight, with respect to the total weight of the composition, preferably from 0.1% to 1% by weight, with respect to the total weight of the composition.

Preferably, said non-ionic surfactant is chosen from: esters of fatty acid and of polyol, and in particular glyceryl esters of fatty acid, such as the ester of fatty acid PEG-6 caprylic/capric glycerides; polycondensates, in particular polymers having oxyethylene and oxypropylene blocks, such as poloxamers; sorbitan fatty acid esters, such as sorbitan monostearate, and their oxyethylenated derivatives, such as polysorbates;
oxyethylenated ethers of dihydrocholesterol; alkyl polyglycosides, in particular alkyl polyglucosides (APGs) having an alkyl group comprising from 6 to 30 carbon atoms and preferably from 8 to 16 carbon atoms, such as, for example, decyl glucoside, caprylyl/capryl glucoside, lauryl glucoside and coco glucoside, and their mixtures.

More preferably, said non-ionic surfactant comprises at least one poloxamer, that is to say a polyethylene glycol/polypropylene glycol/polyethylene glycol triblock polycondensate, preferably with the following chemical structure:

H—(O—CH₂—CH₂)ₐ—(O—CH(CH₃)—CH₂)_{b}—(O—CH₂—CH₂)ₐ—OH, in which formula a ranges from 2 to 150 and b ranges from 1 to 100; preferably, a ranges from 10 to 130 and b ranges from 20 to 80.

More preferably still, the poloxamer is the condensate of ethylene oxide, of propylene oxide and of ethylene oxide (13 EO/30 PO/13 EO) (MW: 2900), in particular such as sold under the name Synperonic PE/L 64 NAA LQ by Croda (INCI name: Poloxamer 184).

Preferably, the concentration of poloxamer (i.e. of active material) in the composition according to invention is within the range from 0.05% to 2% by weight, with respect to the total weight of the composition, and preferably from 0.1% to 1% by weight and more particularly still from 0.2% to 0.9% by weight.

Preferably, the composition according to the invention comprises a mixture of at least one amphoteric surfactant of betaine type, such as those mentioned above, and at least one non-ionic surfactant, such as those preferred and mentioned above.

Preferably, the mixture comprises at least coco betaine and a poloxamer, preferably the condensate of ethylene oxide, of propylene oxide and of ethylene oxide (13 EO/30 PO/13 EO) (MW: 2900), in particular such as sold under the name Synperonic PE/L 64 NAA LQ by Croda (INCI name: Poloxamer 184).

Advantageously, the composition according to the invention comprises at least one first non-ionic surfactant chosen from poloxamers and at least one second non-ionic surfactant chosen from: esters of fatty acid and of polyol; sorbitan fatty acid esters, such as sorbitan monostearate, and their oxyethylenated derivatives, such as polysorbates; alkyl polyglycosides, in particular alkyl polyglucosides (APGs) having an alkyl group comprising from 6 to 30 carbon atoms and preferably from 8 to 16 carbon atoms, such as, for example, decyl glucoside, caprylyl/capryl glucoside, lauryl glucoside and coco glucoside; preferably, said non-ionic surfactant(s) comprise(s) a poloxamer and a polysorbate.

According to a preferred embodiment of the invention, the composition contains a PHA, an AHA, an amphoteric surfactant of betaine type and at least one non-ionic surfactant, in which the content by weight of PHA is greater than the content by weight of AHA, itself greater than the content by weight of amphoteric surfactant of betaine type, and the non-ionic surfactant comprises at least one poloxamer; preferably, said composition contains gluconolactone, glycolic acid, coco betaine, a poloxamer and polysorbate; preferably, the aqueous composition according to the invention comprises from 0.1% to 2% of gluconolactone, from 0.1% to 1% of glycolic acid, from 0.1% to 1% of coco betaine, from 0.1% to 1% of poloxamer and optionally from 0.1% to 1% of polysorbate, by weight, with respect to the total weight of the composition.

### Additional Surfactants

The composition according to the invention can optionally additionally comprise one or more other surfactants chosen from anionic, cationic or amphoteric surfactants (different from the amphoteric surfactants of betaine type described above), and their mixtures.

It can be a hydrocarbon or silicone surfactant and can have, at 25° C., an HLB (hydrophilic-lipophilic balance) in the Griffin sense preferably of greater than or equal to 8. The HLB value according to Griffin is defined in J. Soc. Cosm. Chem., 1954 (Volume 5), pages 249-256. Reference may be made to the document Kirk-Othmer's Encyclopedia of Chemical Technology, Volume 22, pages 333-432, 3rd edition, 1979, Wiley, for the definition of the emulsifying functions and properties of surface-active agents, in particular pages 347-377 of this reference.

Preferably, said additional surfactant is chosen from:
a) anionic surfactants, such as:
   salts of polyoxyethylenated fatty acids, in particular those deriving from alkaline salts, and their mixtures;
   phosphoric esters and their salts, such as DEA oleth-10 phosphate (Crodafos N ION from Croda) or monopotassium monocetyl phosphate (Amphisol K from Givaudan);
   sulfosuccinates, such as disodium PEG-5 citrate lauryl sulfosuccinate and disodium ricinoleamido MEA sulfosuccinate;
   alkyl ether sulfates, such as sodium lauryl ether sulfate;
   isethionates;
   acylglutamates, such as disodium hydrogenated tallow glutamate (Amisoft HS-21 R from Ajinomoto) and sodium stearoyl glutamate (Amisoft HS-11 PF from Ajinomoto) and their mixtures;
   soybean derivatives, such as potassium soyate;
   citrates, such as glyceryl stearate citrate (Axol C 62 Pellets from Degussa);
   proline derivatives, such as sodium palmitoyl proline (Sepicalm VG from SEPPIC) or the mixture of sodium palmitoyl sarcosinate, magnesium palmitoyl glutamate, palmitic acid and palmitoyl proline (Sepifeel One from SEPPIC);
   lactylates, such as sodium stearoyl lactylate (Akoline SL from Karlshamns AB);
   sarcosinates, such as sodium palmitoyl sarcosinate (Nikkol sarcosinate PN) or the 75/25 mixture of stearoyl sarcosine and myristoyl sarcosine (Crodasin SM from Croda);
   sulfonates, such as sodium C14-17 sec-alkyl sulfonate (Hostapur SAS 60 from Clariant);
   glycinates, such as sodium cocoyl glycinate (Amilite GCS-12 from Ajinomoto);
   salts of C₁₆-C₃₀ fatty acids, in particular those deriving from amines, such as triethanolamine stearate and/or 2-amino-2-methylpropane-1,3-diol stearate;
b) amphoteric or zwitterionic surfactants, for instance N-acylamino acids, such as N-alkylaminoacetates (for instance trimethylglycine), disodium cocoamphodiacetate, amine oxides, such as stearamine oxide, or also silicone surfactants, for instance dimethicone copolyol phosphates, such as that sold under the name Pecosil PS 100® by Phoenix Chemical;
c) cationic surfactants, such as optionally polyoxyalkylenated primary, secondary or tertiary fatty amine salts, quaternary ammonium salts and their mixtures; and
(d) their mixtures.

Preferably, the total content of surfactants (i.e. of active material) generally ranges from 0.05% to 10% by weight, with respect to the total weight of the composition, and preferably from 0.1% to 5% by weight and more particularly still from 0.4% to 2% by weight. This is because such a content of surfactants contributes in particular to the formation of micelles within the composition. These micelles can be demonstrated by physicochemical methods, such as the XRD or DLS optical method.

Advantageously, the composition according to the invention is provided in the form of a micellar water; in particular characterized in that it contains free micelles, detectable in particular by DLS (dynamic light scattering) spectroscopic analysis based on the principle of dynamic scattering of light, said micelles preferably exhibiting a median diameter D50 within the range from 1 to 40 nm, preferably from 2 to 30 nm, preferably from 3 to 20 nm, preferably from 5 to 10 nm.

Advantageously, the total content by weight of surfactant(s) in the composition corresponds to a concentration of surfactant(s) of greater than the critical micelle concentration (CMC) of said surfactant(s) in the composition, said CMC preferably being determined by tensiometry.

### Other Ingredients

The composition can also comprise ingredients commonly used in cosmetics, such as additional active principles, antioxidants, preservatives, fragrances, neutralizing agents or their mixtures. Of course, a person skilled in the art will take care to choose the optional additional additives and/or their amount such that the advantageous properties of the composition according to the invention are not, or not substantially, detrimentally affected by the envisaged addition.

Advantageously, the pH of the aqueous composition according to the present invention is of between 4 and 7, preferably within the pH range from 4.5 to 5, that is to say close to the physiological pH: neither too acidic, to avoid any aggressive action on the skin or eyes, nor too basic or neutral (7) because, above pH 6, the peeling effect is only slightly or not observed.

According to one embodiment, the cosmetic composition according to the invention can comprise a neutralizing agent, such as an acid and/or a base.

According to an alternative form, the composition according to the invention can comprise at least one base. The base can be chosen from inorganic bases, such as, for example, alkali metal hydroxides, such as sodium hydroxide, potassium hydroxide, ammonium hydroxides, aqueous ammonia, organic bases, such as, for example, monoethanolamine, diethanolamine, triethanolamine, triisopropylamine, tri(2-hydroxy-1-propyl)amine, N,N-dimethylethanolamine, 2-amino-2-methyl-1-propanol, 2-amino-2-methyl-1,3-propanediol, triethylamine, dimethylaminopropylamine and amphoteric bases (that is to say, bases having both anionic and cationic functional groups), such as primary, secondary, tertiary or cyclic organic amines, amino acids. Mention may be made, as examples of amphoteric bases, of glycine, lysine, arginine, taurine, histidine, alanine, valine, cysteine, trihydroxymethylaminomethane (TRISTA), triethanolamine and any one of their mixtures.

According to a specific embodiment, the base of the composition is chosen from sodium hydroxide, potassium hydroxide, ammonium hydroxides, aqueous ammonia, monoethanolamine, diethanolamine, triethanolamine, tromethamine and any one of their mixtures. According to a specific embodiment, the base of the composition is chosen from sodium hydroxide, triethanolamine and their mixture.

According to a specific embodiment, the base of the composition according to the invention is present according to a content by weight of less than 0.5%, preferably of less than 0.4%, by weight, with respect to the total weight of the composition.

According to an alternative form, the composition according to the invention can comprise at least one additional acid, other than the acids defined above. The acid can be chosen from inorganic acids, such as hydrochloric acid, sulfuric acid or nitric acid, organic acids, such as acetic acid or ascorbic acid, and any one of their mixtures. The acid can be chosen from organic acids, such as benzoic acid, anisic acid and any one of their mixtures.

According to a specific embodiment, the additional acid of the composition according to the invention is present according to a content by weight of less than 1%, preferably of less than 0.5%, by weight, with respect to the total weight of the composition.

The composition according to the invention can be applied by any means making possible uniform distribution over the skin and in particular using a cotton wool swab, a rod, a brush, a gauze, a spatula or a pad, or also by spraying, and it may or may not be removed. It may be removed, for example, by rinsing with water or by simple wiping.

The compositions according to the invention are provided in the aqueous formulation forms usually used in the cosmetic and dermatological fields, preferably in the form of aqueous solutions or dispersions, or optionally in the form of a two-phase composition with an oily phase.

Preferably, the composition according to the invention is provided in the form of a micellar water and/or of a two-phase product. The term “two-phase product” is understood to mean a two-phase composition consisting of an aqueous phase and of an oily phase which are distinct and which are not emulsified in one another at rest. Preferably, the composition according to the invention is provided in the form of a, preferably single-phase, micellar water.

Advantageously, the composition according to the present invention does not require rinsing, in particular with water. In particular, another advantage of the composition according to the present invention is that it is not a foaming composition and thus does not require water, either during the use of the composition or after its use, since it is not necessary to rinse it.

Advantageously, the composition according to the present invention contains less than 2% by weight, preferably less than 1% by weight, preferably less than 0.5% by weight, of sulfate-based surfactant, with respect to the total weight of the composition representing 100%, and preferably the composition according to the present invention is devoid of sulfate-based surfactant, such as sodium laureth sulfate.

The composition of the invention can also contain adjuvants normal in the cosmetic or dermatological field, such as hydrophilic or lipophilic gelling agents, hydrophilic or lipophilic active principles, preservatives (for example phenoxyethanol and parabens), antioxidants, solvents, fragrances, fillers, bactericides, odour absorbers or colorants. The amounts of these various adjuvants are those conventionally used in the field under consideration, for example from 0.01% to 20% of the total weight of the composition. These adjuvants, depending on their nature, can be introduced in the aqueous phase, in an optional oily phase, or dissolved in the surfactants.

Preferably, the composition according to the invention is substantially devoid of hydrophilic polymer, that is to say water-soluble or water-dispersible polymer; that is to say that it contains less than 1% by weight of hydrophilic polymer, indeed even better still the composition according to the invention is completely devoid of hydrophilic polymers, such as cellulose derivatives (carboxymethylcellulose, hydroxyethylcellulose, hydroxypropylmethylcellulose); polycarboxyvinyl polymers of the Carbomer type, such as those sold by Goodrich under the names Carbopol 940, 951 or 980 or by 3V-Sigma under the name Synthalen K or Synthalen L; acrylic copolymers, such as the acrylate/alkyl acrylate copolymers sold under the Pemulen names by Goodrich; polyacrylamides and acrylamide copolymers, such as the product sold under the name Sepigel 305 by SEPPIC, the product sold under the name Hostacerin AMPS by Clariant or the copolymers sold under the Aristoflex names by Clariant.

Preferably, the composition according to the invention is substantially devoid of oily phase (also referred to as fatty phase). The term “substantially devoid of oily phase” is understood to mean that the composition according to the invention exhibits a content of oily phase of less than or equal to 2% by weight, preferably of less than or equal to 1% by weight, with respect to the total weight of the composition, preferably of less than or equal to 0.5% by weight, preferentially of less than or equal to 0.2% by weight, indeed even of less than or equal to 0.1% by weight. Advantageously, the composition according to the invention is completely devoid of oily phase.

The term “oil” is understood to mean a fatty substance which is provided in the liquid form at ambient temperature (20 to 25° C.) and at atmospheric pressure (760 mmHg). The “fatty substance” comprises at least one “fatty” hydrocarbon chain, that is to say a linear hydrocarbon chain of at least 4 carbon atoms which is saturated or unsaturated and optionally substituted, and in particular a linear C₅-C₃₀, preferably C₈-C₃₀, hydrocarbon chain.

When it is present in the composition according to the invention, the oily phase comprises at least one oil. Preferably, the oily phase of the composition according to the invention comprises at least one vegetable oil. The oil(s) of the oily phase is(are) preferably chosen from jojoba seed oil, sweet almond oil, deodorized refined white meadowfoam seed oil, avocado oil, castor oil, olive oil, sunflower oil, wheat germ oil, sesame oil, peanut oil, grape seed oil, soybean oil, rapeseed oil, safflower oil, coconut oil, maize oil, hazelnut oil, palm oil, apricot kernel oil and calophyllum oil, and their mixtures.

The oily phase can also comprise at least one volatile non-polar hydrocarbon oil, preferably chosen from hydrocarbon oils having from 8 to 28 carbon atoms and in particular branched C₁₅-C₁₉ alkanes, such as those sold by SEPPIC under the name Emogreen L19.

The oily phase can also comprise at least one oil chosen from volatile linear alkanes comprising from 7 to 17 carbon atoms, such as undecane, tridecane, C₈-C₁₆ isoalkanes, like isododecane, isodecane or isohexadecane.

In addition, the oily phase can comprise a fatty acid ester, preferably of natural origin. Mention may in particular be made, as fatty acid ester, of synthetic esters of fatty acids, such as the oils of formula R₁COOR₂ in which R₁ represents the residue of a linear or branched higher fatty acid comprising from 1 to 40 carbon atoms and R₂ represents a hydrocarbon chain, in particular a branched hydrocarbon chain, comprising from 1 to 40 carbon atoms with R₁+R₂>10, such as purcellin oil (cetostearyl octanoate), isononyl isononanoate, isopropyl myristate, isopropyl palmitate, C₁₂ to C₁₅ alkyl benzoate, hexyl laurate, diisopropyl adipate, 2-ethylhexyl palmitate, 2-octyldodecyl stearate, 2-octyldodecyl erucate, isostearyl isostearate, tridecyl trimellitate; octanoates, decanoates or ricinoleates of alcohols or of polyalcohols, such as propylene glycol dioctanoate; hydroxylated esters, such as isostearyl lactate, octyl hydroxystearate, octyldodecyl hydroxystearate, diisostearyl malate or triisocetyl citrate; heptanoates, octanoates, decanoates of fatty alcohols; polyol esters, such as propylene glycol dioctanoate, neopentyl glycol diheptanoate, di-ethylene glycol diisononanoate; and esters of pentaerythritol, such as pentaerythrytyl tetraisostearate. Preferably, the fatty acid ester is chosen from isopropyl palmitate, isopropyl myristate, isononyl isononanoate, C₁₂-C₁₅ alkyl benzoate and their mixtures; advantageously, the fatty acid ester is isopropyl palmitate.

The oily phase can additionally comprise conventional cosmetic adjuvants, such as fragrances.

Preferably, when it is present in the composition according to the invention, the oily phase forms, with the aqueous phase, two distinct phases which are not emulsified in one another at rest, and the composition according to the invention is then preferably in the form of a “two-phase composition”. Two-phase compositions differ from emulsions in that, at rest, the two phases are distinct instead of being emulsified in one another. The use of these two-phase compositions requires prior stirring in order to form an emulsion, the latter having to be of sufficient quality and sufficient stability to make possible homogeneous application of the two phases to the skin or the keratin material where it is applied. At rest, said phases must separate rapidly and return to their initial state, this phenomenon being better known under the term “phase separation” or “demixing”. Advantageously, in this case, the aqueous phase/oily phase ratio by weight of the two-phase product according to the invention is within the range from 95/5 to 55/45, preferably within the range from 95/5 to 65/35, preferably from 90/10 to 70/30, preferably from 90/10 to 80/20.

This alternative with optional oily phase nevertheless remains the least preferred since, as indicated above, the composition according to the invention is preferably substantially devoid of oily phase, the composition according to the invention exhibiting a content of oily phase preferably of less than or equal to 2% by weight.

Advantageously, the composition of the present invention is transparent.

The term “transparent composition” is understood to mean, within the meaning of the present invention, a composition exhibiting a turbidity value of less than 20 NTU, preferably of less than 15 NTU, preferably of less than 10 NTU. Preferably, the turbidity of the compositions is at least equal to 1 NTU. Preferably, the turbidity of the compositions is within the range from 0.1 to 10 NTU and preferably from 1 to 10 NTU. The NTU (nephelometric turbidity unit) is the unit of measurement of the turbidity of a composition. The measurement of the turbidity is carried out, for example, with a 2100P model turbidimeter from Hach, the tubes used for the measurement being referenced AR397A cat 24347-06. The measurements are carried out at ambient temperature (from 20° C. to 25° C.). Preferably, the composition is transparent and exhibits a turbidity value of between 1 and 20 NTU, preferably between 1 and 15 NTU, preferably of less than 10 NTU.

Preferably, the composition according to the invention is packaged in a transparent UV-resistant packaging.

The present invention also relates to the use of the composition as defined above for removing make-up from and/or cleansing keratin materials, preferably skin and/or mucous membranes (such as the lips) and/or keratin fibres (such as eyelashes).

Another subject-matter of the present invention is a method for removing make-up from and/or for cleansing keratin materials, preferably skin and/or mucous membranes (such as lips, outline of the eyes or eyelids) and/or keratin fibres (such as eyelashes), comprising the application, to the keratin materials, of a composition according to the invention. Preferably, just after a method for removing make-up with an even mild peeling effect, it is recommended to avoid exposure to the sun. Advantageously, the method according to the invention thus additionally comprises at least one stage of application of a UV inhibitor, preferably with an SPF (sun protection factor) at least equal to SPF 30, preferably the morning after use of the composition according to the present invention.

Throughout the patent application, the wording “comprising a” or “comprising an” means “comprising at least one”, unless otherwise specified.

The invention is now illustrated by the following non-limiting examples. The per-centages are expressed by weight (of active material), with respect to the total weight of composition (% w/w), unless otherwise mentioned.

### EXAMPLES

The aqueous compositions described in detail in the tables below are prepared according to the conventional methods of a person skilled in the art. The ingredients of the aqueous phase are mixed and the mixture is heated to the vicinity of 50° C. to 60° C. Then the optional salts (NaOH, NaCl) are added and finally the preservatives.

TABLE 1

| INCI | Ex. 25 | Ex. 30 |
|---|---|---|
| Water | 93.76 | 93.76 |
| Glycerol | 1.00 | 1.00 |
| Hexylene glycol | 2.00 | 2.00 |
| Gluconolactone | 1.00 | 1.00 |
| Glycolic acid | 0.41 | 0.41 |
| Coco betaine | 0.15 | |
| Disodium Cocoamphodiacétate | | 0.15 |
| Poloxamer 184 | 0.50 | 0.50 |
| Polysorbate 20 | 0.50 | 0.50 |
| Sodium hydroxide | 0.38 | 0.38 |
| Sodium chloride | 0.03 | 0.03 |
| Disodium EDTA | 0.20 | 0.20 |
| Myrtrimonium bromide | 0.07 | 0.07 |
| turbidity value (20-25° C.) | <10 NTU | <20 NTU |
| turbidity value (12° C.) | <10 NTU | >20 NTU |
| turbidity value (4° C.) | <10 NTU | >20 NTU |

Both formulas present a pH 4.8.

1—Evaluation of Cell Renewal by Labelling the Skin with Dansyl Chloride

Principle: The movement of cells through the stratum corneum of the epidermis to the surface of the skin is a function of the rate of production of new cells. It has been reported that dansyl chloride binds strongly to amino acids and has become useful as a fluorescent label for proteins. The disappearance of the fluorescent marker from the stratum corneum is an indicator of the movement of cells through the epidermis and can thus be regarded as a measure of the rate of cell proliferation, often called cell renewal.

Protocol: Subjects: N=29 women in good health aged from 39 to 63 years; Duration of the study: 22 days; Measurement points taken at the following evaluation times: Days: D3, D5, D7 and D22

Design of the tests: Two patches of 3.5×3.5 cm containing 0.2 g of dansyl chloride (5% in petroleum jelly) are applied to a randomized palmar forearm. The patches will be left in place for 24 hours in order to make it possible for the skin to be saturated with dansyl chloride. After the withdrawal of the patch, the reference level of fluorescence is evaluated in a dark room under lighting with a Wood's lamp.

The subjects apply the test product to the assigned area (avoiding the untreated control area) on the palmar forearm once daily in the evening for the duration of the study. A cotton disc saturated with the product (the composition) to be tested (test material) is used on the test site. The test material is applied by gently wiping a saturated cotton disc over the designated test site on the palmar surface of the right or left forearm. The (untreated) control site of the forearm remains untreated throughout the study and is wiped with cotton discs saturated with water at the same frequency as for the test material.

The subjects return for a fluorescence evaluation every day for 22 consecutive days (less the weekends). The cumulative mean scores of fluorescence for the treated and untreated sites were tabulated on days 3, 5, 7 and 22 and compared via a statistical analysis: a P≤0.05 value indicates that the result is significant (in bold in the following tables), that is to say that a statistically significant improvement is obtained compared with the control.

Formula tested: Ex. 25 at Day 5, Day 7 and Day 23.

The total cumulative scores of fluorescence for the formula Ex. 25 (“treated” site) and the reference or “control” value (“untreated” site) are shown in the table below, then the P value obtained according to the Wilcoxon signed-rank test method indicates if the result obtained is significant.

TABLE 2

| | | | | | Wilcoxon | Statistical |
|---|---|---|---|---|---|---|
| | | | | | signed-rank | significance |
| Measurement | Treated/untreated | | Standard | | test | (Yes if P ≤ |
| point | (control) site | Mean | deviation | Median | P value | 0.05) |
| Day 3 | treated | 14.57 | 0.50 | 15.00 | 0.3458 | No |
| | | control | 14.63 | 0.49 | | |
| Day 5 | | treated | 22.38 | 1.76 | 22.50 | 0.0011 |
| | | control | 22.97 | 1.31 | 22.50 | |
| Day 7 | | treated | 29.57 | 3.19 | 29.00 | 0.0011 |
| | | control | 30.90 | 2.31 | 30.00 | |
| Day 23 | | treated | 48.67 | 5.96 | 48.00 | <0.001 |
| | | control | 53.70 | 5.06 | 52.00 | |

The table below indicates the number of days for cell renewal at the site “treated” by the formula according to the present invention Ex. 25 compared with the untreated (“control”) site:

TABLE 3

| | | | | Wilcoxon |
|---|---|---|---|---|
| Treated/untreated | | Standard | | signed-rank |
| (control) test site | Mean | deviation | Median | test P value |
| treated | 9.93 | 2.15 | 9.50 | <0.001 |
| control | 12.43 | 2.70 | 11.00 | |

Table 3 shows that the cell renewal at the site “treated” by the formula according to the present invention Ex. 25 is faster than that of the untreated (“control”) site.

Table 4 below displays the area under the curve (AUC) results, graphically representing the daily fluorescence dye score over the entire duration of the study.

TABLE 4

| | | | | The paired |
|---|---|---|---|---|
| Treated/untreated | | Standard | | t-test |
| (control) test site | Mean | deviation | Median | P value |
| treated | 45.67 | 5.96 | 45.00 | <0.001 |
| control | 50.70 | 5.06 | 49.00 | |

It has thus been demonstrated that this score (AUC) at the treated site was lower than the score (AUC) obtained for the control site, with a significant difference.

### Conclusion of the Cell Renewal Test:

Table 2 shows the total fluorescence scores at days 3, 5, 7 and 23. A lower total score represents a decrease in the fluorescence marker of dansyl chloride of the stratum corneum, and thus signals the moment of cell renewal. The treated sites showed lower total cumulative scores for fluorescence (according to a statistically significant difference) at day 5, at day 7 and at day 23, compared with the untreated control site. Table 3 shows that the treated sites have a statistically significant faster cell renewal rate (in days), compared with the untreated control site. Finally, the area under the curve (AUC), shown in Table 4, corresponds to the daily fluorescence dye score over the entire duration of the study. It has also being demonstrated that the AUC for the treated site was lower (significant difference) than the AUC at the control site.

Thus, the composition according to the present invention, tested under the conditions of this study, helps to accelerate cell renewal, compared with the untreated control site.

### 2—Measurement of the Insensible Water Loss or Transepidermal Water Loss (TEWL)

The effectiveness of the formula Ex. 25 with regard to the transepidermal water loss was objectively evaluated by biometrological measurement.
Panel: 24 women aged 27 to 65 years (mean=54 years), with an TEWL≥6 g·m⁻²·h⁻¹ value on the anterior face of the forearms.
Treatment: The product was applied in one go, with a single-use fingerstall, by the technician to a defined area of the forearm, in an amount of 2 mg/cm².
Evaluation: A Tewameter® of Courage & Khazaka trademark was used as measuring device for the evaluation of the rate of transepidermal water loss (TEWL).

The measurement is made at T0 and T1h after application of the product.

### Results:

The mean rate of improvement in the TEWL in % is obtained by the following calculation method:

$$\% mean improvement = \vert \frac{( {T}_{i} - {T}_{0} ) P}{{T}_{0} P} \times 100 ] - [ \frac{( {T}_{i} - {T}_{0} ) Te}{{T}_{0} Te} \times 100 ]$$

$$With :$$

$${T}_{0} = mean of TEWL levels values at T 0$$

$${T}_{i} = mean of TEWL level values at Ti$$

$$P = Product$$

$$Te = Control ( non treated area )$$

in which T0: mean of the values of level of TEWL at T0; Ti: mean of the values of level of TEWL at Ti (i=1 h in the present example); “P”: the measurement is relative to the tested product/to the site treated by the tested product; “Te”: the measurement is relative to the control/to the untreated site.

TABLE 5

| | | | P value | Mean | | |
|---|---|---|---|---|---|---|
| | | | (mean | change | | |
| | | | value at | with | | |
| | | | the | respect to | | |
| | | Mean over 24 | treated | the | Mean rate of | |
| | | measurements ± | site vs. | baseline | improvement | |
| | | standard | untreated | (±standard | in the | P value |
| N = 24 | Site | deviation | site) | deviation) | TEWL in % | (t-Test) |
| Baseline, T0 | treated | 9.08 ± 1.73 | p = 0.346 | | | |
| | untreated | 9.34 ± 1.95 | NS/not | | | |
| | | | significant | | | |
| T1 hour | treated | 8.12 ± 2.08 | | −0.96 ± 0.79 | −6.8% | p = 0.002 |
| | | | | | | S/significant |
| | untreated | 8.98 ± 1.99 | | −0.36 ± 0.36 | | |

Conclusion: The formula according to the invention Ex. 25 significantly reduces the TEWL (insensitive water loss or transepidermal water loss) at the treated site compared with the control site (untreated), 1 hour after application. The composition of the present invention exhibits a dermoprotective effect.

### 3—Test of Effectiveness Perceived by Consumers

Tested formula: Ex. 25

### Protocol:

Panel of 150 people; Duration of the test: 42 days; Frequency of application: twice daily, morning and evening; Application sites: on the face, the eyes and the lips.

Method of employment used: “Use this product on the face, the eyes and the lips, twice daily, in the morning as a cleanser and in the evening as a make-up remover, for 42 days, instead of your usual cleanser and/or your make-up remover. Start using the product overnight. Apply the SPF 30 care formulation (provided) in the morning, after having used the product.”

The scale used by the panellists is a scale from 1 to 5, thus in 5 steps: 1. Agree; 2. Somewhat agree; 3. Neither agree nor disagree; 4. Somewhat disagree; 5. Disagree

The results obtained (mean of the responses over 150 panellists) (“% Agree”) are given in %, in the following table. Any result greater than 60% is regarded as a significant result.

TABLE 6

| | Immediately | | | |
|---|---|---|---|---|
| | after the 1st | Immediately | | |
| | use, in the | after the 2nd | | |
| Questions: | evening as a | use, in the | | |
| (the means of the respective | make-up | morning as a | After | After |
| responses are given in %): | remover | cleanser | 21 days | 42 days |
| The skin appears cleansed: | 92.7% | 95.3% | 96.6% | 94.9% |
| The skin is clean: | 94.7% | 94.7% | 94.6% | 96.4% |
| The product removes all the | 84.7% | | 89.8% | 92.0% |
| impurities (oil, dirt, dust, pollution): | | | | |
| The product removes make-up from | 90.6% | | 91.5% | 91.8% |
| the lips: | | | | |
| The product removes make-up from | 95.2% | | 90.2% | 91.1% |
| the face: | | | | |
| The product removes make-up from | 88.2% | | 88.1% | 88.3% |
| the eyes: | | | | |
| The skin is smooth: | 66.7% | 71.3% | 75.5% | 78.8% |
| The skin is soft: | 73.3% | 77.3% | 81.6% | 87.6% |
| The skin texture appears finer: | 42.0% | 53.3% | 61.9% | 68.6% |
| The complexion is freshened: | 74.0% | 74.7% | 81.6% | 86.1% |
| After application, no product residue | 91.3% | 90.7% | | 92.0% |
| is visible on the skin: | | | | |
| The pores seem clear: | 48.0% | | 73.5% | 74.5% |
| The skin is soothed: | 62.0% | 70.7% | 76.9% | 76.6% |
| The skin tugs less: | 74.0% | 66.0% | 67.3% | 73.9% |
| The skin appears healthier: | 68.7% | 73.3% | 81.0% | 84.7% |
| The complexion is more uniform: | 41.3% | 56.0% | 64.6% | 74.5% |
| The skin appears less dull: | | | 64.6% | 73.7% |
| The skin is gently exfoliated: | 44.0% | 54.7% | 64.6% | 74.5% |
| The skin appears purified, as | 58.0% | 69.3% | 76.2% | 80.3% |
| detoxified: | | | | |
| The skin is regenerated, renewed: | | | 62.6% | 68.6% |
| The skin is soft like a baby's skin: | 46.7% | 55.3% | 63.9% | 67.9% |
| The skin is not tacky and greasy after | | | | 95.6% |
| application: | | | | |
| The product is gentle on the skin: | | | | 85.4% |
| The product does not dry out the | | | | 81.8% |
| skin: | | | | |
| The product is suitable for my skin | | | | 86.9% |
| type: | | | | |

### 4—Clinical Test of Ocular and Cutaneous Tolerance

The cutaneous tolerance and the ocular tolerance of a cosmetic product according to the present invention (formula Ex. 25) were evaluated by a test of use of the product under dermatological and ophthalmological controls, respectively.

Protocol: The panel used consisted of 51 people; 6 men, 45 women; aged between 19 and 65 years; of all skin types; 49% of them having stated that they had sensitive skin, 45% that they had sensitive eyes and 14% that they wore contact lenses. Duration of the test: 4 weeks; Number of applications per day of the product: twice daily; Application sites: on the face, the eyes and the lips.

### Results:

Dermatological result: very well tolerated product.
Ophthalmological result: very well tolerated product.
94% of the subjects stated that the product was suitable for the skin.

### 5—Test of Effectiveness for the Peeling Effect or Desquamating Effect

In this test, the peeling effect is related to the ability of the sample to induce corneocytes by unsticking or detachment, at D13 (13 days) after topical treatment on Episkin® reconstructed skin (single application, treatment over 20 min). The detachment of the corneocytes is indirectly quantified by measuring the concentration of total proteins in the rinsing liquid (BCA assay). The desquamation potential (detachment of the corneocytes) was evaluated on several formulas and compared with the effect of a 30% aqueous solution of glycolic acid (the reference). The responses are measured in g/ml of proteins coming from the detached corneocytes. Three batches of Episkin are used for each study (n=3 per batch and per condition).

The evaluation of the peeling effect of the formulae of Table 7 is done by comparing the results of the formula tested with that obtained with 30% glycolic acid in water (Ref.) forming the reference line representing 100% effectiveness.

TABLE 7

| INCI | Ref. | Cp. 3 | Cp. 5 | Cp. 7 | Cp. 13 | Ex. 15 | Ex. 16 |
|---|---|---|---|---|---|---|---|
| Water | 70 | 98.81 | 97.37 | 97.08 | 95.19 | 94.10 | 93.17 |
| Glycerol | | 1.00 | 1.00 | 1.00 | 1.06 | 1.06 | 1.06 |
| Gluconolactone | | | | 1.00 | | 1.00 | 1.00 |
| Glycolic acid | 30 | | 1.00 | 0.41 | 0.41 | 0.41 | 1.00 |
| Coco betaine | | 0.15 | 0.15 | 0.15 | 0.15 | 0.15 | 0.15 |
| Poloxamer 184 | | | | | 0.50 | 0.50 | 0.50 |
| Polysorbate 20 | | | | | 0.50 | 0.50 | 0.50 |
| Sodium hydroxide | | | 0.45 | 0.33 | 0.16 | 0.25 | 0.58 |
| Sodium chloride | | 0.03 | 0.03 | 0.03 | 0.03 | 0.03 | 0.03 |
| Peeling effect (% Ref.) | 100 | 40 | 45 | 42 | 55 | 82 | 60 |

Results: A peeling effect of at least 60% was demonstrated with a micellar water according to the invention (Ex. 15, Ex. 16) combining PHA (gluconolactone), AHA (glycolic acid), amphoteric surfactant of betaine type (coco betaine) and non-ionic surfactants (poloxamer 184, polysorbate 20). The tested formula Ex. 15 is the most effective for a desquamating effect which reaches at least 80% of the reference level. In particular, with Ex. 15, a peeling effect of greater than 80% was demonstrated with a micellar water according to the invention combining 1% of gluconolactone, 0.41% of glycolic acid, 0.5% of poloxamer and 0.5% of polysorbate 20. On the contrary, for the other formulae not in accordance with the invention (Cp. 3 to Cp. 7), the peeling effect is insufficient because it is less than 50% of the reference level.

### 6—Evaluation of the Impact of Different Ingredients on the Peeling Effect

Test used: As in the preceding test, the peeling or desquamating effect is related to the ability of the sample to induce the detachment of the corneocytes after topical treatment on a D13 Episkin reconstructed epidermis. The detachment of the corneocytes is indirectly quantified by the measurement of the concentration of total proteins in the rinsing liquid (BCA assay). The objective is to evaluate the effectiveness of poloxamer 184, of hexylene glycol and of polysorbate 20, tested alone or according to different combinations, on the peeling effect.

The peeling effect (detachment of the corneocytes) was evaluated on several formulae and three simplexes (% in water) described in Table 8 below:

TABLE 8

| INCI US | Ref. | Ex. 18 | Ex. 20 | Ex. 22 | Ex. 24 | Cp. 27 | Cp. 28 | Cp. 29 |
|---|---|---|---|---|---|---|---|---|
| Water | 70 | 96.50 | 96.50 | 96.00 | 94.00 | 99.50 | 98.00 | 99.5 |
| Glycerol | | 1.06 | 1.06 | 1.06 | 1.06 | | | |
| Hexylene glycol | | | | | 2.00 | | 2.00 | |
| Gluconolactone | | 1.00 | 1.00 | 1.00 | 1.00 | | | |
| Glycolic acid | 30 | 0.41 | 0.41 | 0.41 | 0.41 | | | |
| Coco betaine | | 0.15 | 0.15 | 0.15 | 0.15 | | | |
| Poloxamer 184 | | 0.50 | | 0.50 | 0.50 | 0.50 | | |
| Polysorbate 20 | | | 0.50 | 0.50 | 0.50 | | | 0.50 |
| Sodium hydroxide | | 0.35 | 0.35 | 0.35 | 0.35 | | | |
| Sodium chloride | | 0.03 | 0.03 | 0.03 | 0.03 | | | |
| Peeling effect (% Ref.) | 100 | 92 | 60 | 88 | 95 | 50 | 30 | 52 |

Under the experimental conditions of this study (single application, 20 min):
The peeling effect of the formulae according to the invention (Ex. 18, 20, 22, 24) is confirmed.
No peeling effect was observed with the 3 simplex formulae (Cp. 27, 28, 29): poloxamer 184 (0.5%), hexylene glycol (2%) or polysorbate 20 (0.5%) in water.
The presence of poloxamer in the formulae Ex. 18, 22, 24 boosts the peeling effect, which is of the order of 90% of that of the reference (100%) of 30% glycolic acid in water.

A marked improvement in the peeling effect, indeed even a synergistic effect, is observed by combining the non-ionic surfactant, poloxamer, and the “base of active principles, PHA, AHA, and amphoteric surfactant, coco betaine”, in Examples 18, 22 and 24 according to the invention.
The combination of active principles according to a content by weight PHA>AHA>coco betaine and of poloxamer in the compositions according to the invention not only exceeds the peeling effects of each of the ingredients taken alone (there is a synergy) but above all these formulae exhibit an effectiveness comparable to that of the 30% glycolic acid in water reference.

In conclusion, the combinations tested in accordance with the present invention contribute a peeling advantage comparable to that of the glycolic acid reference.

### 7—Measurement of Micellarity:

Tensiometric data for the formula Ex. 25 showed a decreasing slope of the surface tension and, after a certain concentration, the surface tension no longer changes. The intersection of the slope and of the surface tension plateau indicates the presence of micelles in the formula.

DLS plots showed a peak in the vicinity of 5-10 nm which corresponds to the presence of micelles in the formula Ex. 25, whereas no peak of this type was observed in the placebo.

1. Cosmetic composition, preferably micellar water, which removes make-up from and/or cleanses keratin materials, comprising an aqueous phase comprising:
at least one polyhydroxy acid (PHA),
at least one hydroxy acid chosen from α-hydroxy acids (AHAs) and/or β-hydroxy acids (BHAs),
at least one amphoteric surfactant, preferably of betaine type,
and at least one non-ionic surfactant.

2. Composition according to claim 1, in which:
the ratio by weight of polyhydroxy acid(s) to hydroxy acid(s) (AHA(s) and/or BHA(s)) is greater than 1; and/or
the ratio by weight of polyhydroxy acid(s) to amphoteric surfactant(s) of betaine type is greater than 1; and/or
the ratio by weight of hydroxy acid(s) (AHA(s) and/or BHA(s)) to amphoteric surfactant(s) of betaine type is greater than 1;
preferably, the content by weight of polyhydroxy acid(s) is greater than the content by weight of hydroxy acid(s) (AHA(s) and/or BHA(s)), itself greater than the content by weight of surfactant(s) of betaine type,
by weight, with regard to the total weight of the composition.

3. Composition according to claim 1, in which said polyhydroxy acid is chosen from: dihydroxypropanoic acid, such as glyceric acid; trihydroxybutanoic acid, such as erythronic acid or threonic acid; tetrahydroxypentanoic acid, such as ribonic acid, arabinonic acid, xylonic acid and lyxonic acid; pentahydroxyhexanoic acid, such as allonic acid, altronic acid, gluconic acid, mannonic acid, gulonic acid, idonic acid, galactose, galactonic acid or talonic acid; hexhydroxyheptanoic acid, such as glucoheptanoic acid, galactoheptonic acid; lactobionic acid, maltobionic acid, their derivatives, such as their salts or their lactone forms, such as gluconolactone or ribonolactone, and their mixtures; preferably, the polyhydroxy acid is provided in the form of a lactone, the lactone ring preferably being saturated; preferably, the polyhydroxy acid is chosen from gluconolactone, ribonolactone and their mixtures; preferably, the polyhydroxy acid according to the invention is chosen from: lactobionic acid, galactose, gluconic acid, gluconolactone and their mixtures; preferably, said polyhydroxy acid is gluconolactone.

4. Composition according to claim 1, characterized in that the PHA(s) is(are) present in the composition according to a content within the range from 0.1% to 5%, preferably from 0.2% to 4%, preferably from 0.5% to 3%, preferably from 1% to 2%, by weight (of active material), with respect to the total weight of the composition.

5. Composition according to claim 1, in which:
said α-hydroxy acid(s) (AHA(s)) is(are) chosen from: glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid; mandelic acid; phenyllactic acid; galacturonic acid; aleuritic acid; tartronic acid; tartaric acid; malic acid; fumaric acid; their salts and their mixtures; preferably, the α-hydroxy acid is chosen from glycolic acid, citric acid, malic acid, tartaric acid, lactic acid, mandelic acid and their salts; more particularly, the α-hydroxy acid is chosen from glycolic acid, citric acid, lactic acid, their salts and their mixtures, and preferably said AHA is glycolic acid; and/or
said β-hydroxy acid(s) (BHA(s)) is(are) chosen from: salicylic acid and its derivatives, preferably chosen from salicylic acid, 5-(n-octanoyl)salicylic acid, 5-(n-decanoyl)salicylic acid, 5-(n-dodecanoyl)salicylic acid and 5-(n-heptyloxy)salicylic acid; preferably, said BHA is salicylic acid.

6. Composition according to claim 1, characterized in that the α-hydroxy acid(s) and/or β-hydroxy acid(s) are present according to a content within the range from 0.001% to 10% by weight, preferably from 0.01% to 5% by weight, preferably from 0.1% to 3% by weight and better still from 0.1% to 2% by weight, preferably from 0.1% to 1.5% by weight, preferably from 0.1% to 1% by weight, preferably from 0.2% to 0.8% by weight (of active material), with respect to the total weight of the composition.

7. Composition according to claim 1, in which the amphoteric surfactant of betaine type is chosen from alkyl betaines, such as coco betaine and/or lauryl betaine, and N-alkylamido betaines, such as cocamidopropyl betaine; sultaines, such as cocoylamidopropyl hydroxysulfobetaine; preferably from alkyl betaines; more preferentially, said amphoteric surfactant is coco betaine.

8. Composition according to claim 1, in which the amphoteric surfactant(s) of betaine type is(are) present in the composition in a proportion of 0.1% to 10% by weight, preferably of 0.1% to 5% by weight, preferably of 0.1% to 3% by weight, preferably of 0.1% to 2% by weight, preferably of 0.1% to 1% by weight, preferably of 0.1% to 0.5% by weight, of active material, with respect to the total weight of the composition.

9. Composition according to claim 1, in which said non-ionic surfactant is chosen from: esters of fatty acid and of polyol, and in particular glyceryl esters of fatty acid, such as the ester of fatty acid PEG-6 caprylic/capric glycerides; polycondensates, in particular polymers having oxyethylene oxypropylene blocks, such as poloxamers; sorbitan fatty acid esters, such as sorbitan monostearate, and their oxyethylenated derivatives, such as polysorbates; alkyl polyglycosides, in particular alkyl polyglucosides (APGs) having an alkyl group comprising from 6 to 30 carbon atoms and preferably from 8 to 16 carbon atoms, such as, for example, decyl glucoside, caprylyl/capryl glucoside, lauryl glucoside and coco glucoside, and their mixtures; preferably, said non-ionic surfactant comprises at least one polyethylene glycol/polypropylene glycol/polyethylene glycol triblock polycondensate, preferably with the following chemical structure:
H—(O—CH2-CH2)ₐ—(O—CH(CH3)-CH2)_{b}—(O—CH2-CH2)ₐ—OH, in which formula a ranges from 2 to 150 and b ranges from 1 to 100; preferably, a ranges from 10 to 130 and b ranges from 20 to 80.

10. Composition according to claim 1, in which the non-ionic surfactant(s) is(are) present in the composition according to an active material content ranging from 0.01% to 10% by weight, preferably from 0.05% to 5% by weight, preferably from 0.05% to 2% by weight, with respect to the total weight of the composition, preferably from 0.1% to 1% by weight, with respect to the total weight of the composition.

11. Composition according to claim 1, comprising at least one first non-ionic surfactant chosen from poloxamers and at least one second non-ionic surfactant chosen from: esters of fatty acid and of polyol; sorbitan fatty acid esters, such as sorbitan monostearate, and their oxyethylenated derivatives, such as polysorbates; alkyl polyglycosides, in particular alkyl polyglucosides (APGs) having an alkyl group comprising from 6 to 30 carbon atoms and preferably from 8 to 16 carbon atoms, such as, for example, decyl glucoside, caprylyl/capryl glucoside, lauryl glucoside and coco glucoside; preferably, said non-ionic surfactant(s) comprise(s) a poloxamer and a polysorbate.

12. Composition according to claim 1, characterized in that it comprises at least 50% of water and optionally at least one organic solvent soluble in water at 25° C., advantageously chosen from polyols having in particular from 2 to 20 carbon atoms, preferably from 2 to 6 carbon atoms, such as glycerol, diglycerol, propylene glycol, isoprene glycol, dipropylene glycol, butylene glycol, hexylene glycol, 1,3-propanediol, pentylene glycol, polyethylene glycols having from 2 to 200 ethylene oxide units and their mixtures; preferably, the composition comprises from 50% to 99% by weight of water, with respect to the total weight of the composition, preferably from 70% to 98% by weight, preferably from 80% to 98% by weight, preferably from 85% to 97% by weight, of water and/or from 0.01% to 15% by weight of organic solvent, preferably from 0.5% to 13% by weight, better still from 1% to 10% by weight, with respect to the total weight of the composition.

13. Composition according to claim 1, characterized in that it is single-phase, preferably transparent, preferably colourless, preferably it exhibits the same appearance as water, and in particular the same texture as liquid water at ambient conditions of temperature (25° C.) and of pressure (100 kPa); preferably, the composition contains less than 1% by weight of hydrophilic polymer, indeed even better is completely devoid of hydrophilic polymers, such as cellulose derivatives, in particular carboxymethylcellulose, hydroxyethylcellulose or hydroxypropylmethylcellulose; polycarboxyvinyl polymers of the Carbomer type; acrylic copolymers, such as acrylate/alkyl acrylate copolymers; polyacrylamides and acrylamide copolymers.

14. Composition according to claim 1, characterized in that it exhibits a turbidity of less than or equal to 20 NTU, preferably of less than 15 NTU, preferably of less than 10 NTU; preferably, the turbidity of the compositions is within the range from 0.1 to 10 NTU and preferably from 1 to 10 NTU.

15. Composition according to claim 1, characterized in that it is provided in the form of a micellar water or of a two-phase product, preferably in the form of a micellar water; in particular characterized in that it contains free micelles, detectable in particular by DLS (dynamic light scattering) spectroscopic analysis based on the principle of dynamic scattering of light, said micelles preferably exhibiting a median diameter D50 within the range from 1 to 40 nm, preferably from 2 to 30 nm, preferably from 3 to 20 nm, preferably from 5 to 10 nm.

16. Composition according to claim 1, characterized in that the content by weight of surfactant(s) in the composition corresponds to a concentration of surfactant(s) of greater than the critical micelle concentration (CMC) of said surfactant(s) in the composition, said CMC preferably being determined by tensiometry.

17. Composition according to claim 1, characterized in that the content of fatty substance, in particular of fatty substance which is liquid at ambient temperature (25° C.), such as oil, if the composition contains it, does not exceed 2% by weight, preferably does not exceed 1% by weight, preferably does not exceed 0.5% by weight, preferably does not exceed 0.2% by weight, indeed even does not exceed 0.1% by weight, with respect to the total weight of the composition; preferably, the composition is lacking in, indeed even completely devoid of, fatty substance, in particular liquid fatty substance, such as oil.

18. Composition according to claim 1, characterized in that it contains a PHA, an AHA, an amphoteric surfactant of betaine type and at least one non-ionic surfactant, in which the content by weight of PHA is greater than the content by weight of AHA, itself greater than the content by weight of amphoteric surfactant of betaine type, and the non-ionic surfactant comprises at least one poloxamer; preferably, said composition contains gluconolactone, glycolic acid, coco betaine, at least one poloxamer and a polysorbate; preferably, the aqueous composition according to the invention comprises from 0.1% to 2% of gluconolactone, from 0.1% to 1% of glycolic acid, from 0.1% to 1% of coco betaine, from 0.1% to 1% of poloxamer and optionally from 0.1% to 1% of polysorbate, by weight, with respect to the total weight of the composition.

19. Method for removing make-up from and/or for cleansing keratin materials, preferably skin and/or mucous membranes, such as the lips, eyelids, and/or keratin fibres, such as eyelashes, comprising the application, to the keratin materials, of a composition according to claim 1.

20. Method for removing make-up from and/or for cleansing keratin materials, preferably skin and/or mucous membranes, such as the lips, eyelids, and/or keratin fibres, such as eyelashes, comprising the application, to the keratin materials, of a composition according to claim 2.
